(19) United States
(12) Patent Application Publication
(10) Pub. No.: US 2009/0259194 A1
(43) Pub. Date: Oct. 15, 2009

(54) Keyed Safety Cap and Receptacle for Two Piece Injection Device

(76) Inventors: Raffi Pinedjian, Fountain Valley, CA (US); Robert J. Sanchez, JR., Oceanside, CA (US); Jeffrey Chun, Irvine, CA (US)

Correspondence Address: ALCON, IP LEGAL, TB4-8, 6201 SOUTH FREEWAY, FORT WORTH, TX, 76134, US

(21) Appl. No.: 12/101,595

(22) Filed: Apr. 11, 2008

(51) Int. Cl. A61M 5/32

(52) U.S. Cl. 604/192

(57) ABSTRACT

A keyed safety system for an injection device that has a safety cap and a receptacle. The safety cap has a generally hollow body for receiving the needle end of an injection device. An orientation mechanism is disposed on the outer surface of the safety cap, a retention mechanism is disposed on the outer surface of the safety cap, and a shroud extends from the safety cap. The receptacle has a cavity for receiving the safety cap. A complimentary orientation mechanism is disposed on a surface of the cavity, and a complimentary retention mechanism is disposed on the surface of the cavity. The orientation mechanism on the safety cap is configured to engage the complimentary orientation mechanism on the cavity so that the safety cap fits into the cavity in a single orientation. The retention mechanism on the safety cap is configured to engage the complimentary retention mechanism on the cavity so that the safety cap is prevented from rotating in the cavity.

### BACKGROUND OF THE INVENTION

The present invention relates to a single-use medical device and more particularly to a keyed safety cap and matching receptacle for a two-piece ophthalmic injection device with a disposable tip.

Several diseases and conditions of the posterior segment of the eye threaten vision. Age related macular degeneration (ARMD), choroidal neovascularization (CNV), retinopathies (e.g., diabetic retinopathy, vitreoretinopathy), retinitis (e.g., cytomegalovirus (CMV) retinitis), uveitis, macular edema, glaucoma, and neuropathics are several examples.

These, and other diseases, can be treated by injecting a drug into the eye. Such injections are typically done manually using a conventional syringe and needle. FIG. 1 is a perspective view of a prior art syringe used to inject drugs into the eye. In FIG. 1, the syringe includes a needle 105, a luer hub 110, a chamber 115, a plunger 120, a plunger shaft 125, and a thumb rest 130. As is commonly known, the drug to be injected is located in chamber 115. Pushing on the thumb rest 130 causes the plunger 120 to expel the drug through needle 105.

In using such a syringe, the surgeon is required to pierce the eye tissue with the needle, hold the syringe steady, and actuate the syringe plunger (with or without assistance) to inject the fluid into the eye. The volume injected is typically not controlled in an accurate manner because reading the vernier is subject to parallax error. Fluid flow rates are uncontrolled and tissue damage may occur due to an “unsteady” injection. Reflux of the drug may also occur when the needle is removed from the eye.

An effort has been made to control the delivery of small amounts of liquids. A commercially available fluid dispenser is the ULTRA™ positive displacement dispenser available from EFD Inc. of Providence, R.I. The ULTRA dispenser is typically used in the dispensing of small volumes of industrial adhesives. It utilizes a conventional syringe and a custom dispensing tip. The syringe plunger is actuated using an electrical stepper motor and an actuating fluid. Parker Hannifin Corporation of Cleveland, Ohio distributes a small volume liquid dispenser for drug discovery applications made by Aurora Instruments LLC of San Diego, Calif. The Parker/Aurora dispenser utilizes a piezo-electric dispensing mechanism. Ypsomed, Inc. of Switzerland produces a line of injection pens and automated injectors primarily for the self-injection of insulin or hormones by a patient. This product line includes simple disposable pens and electronically-controlled motorized injectors.

U.S. Pat. No. 6,290,690 discloses an ophthalmic system for injecting a viscous fluid (e.g. silicone oil) into the eye while simultaneously aspirating a second viscous fluid (e.g. perflourocarbon liquid) from the eye in a fluid/fluid exchange during surgery to repair a retinal detachment or tear. The system includes a conventional syringe with a plunger. One end of the syringe is fluidly coupled to a source of pneumatic pressure that provides a constant pneumatic pressure to actuate the plunger. The other end of the syringe is fluidly coupled to an infusion cannula via tubing to deliver the viscous fluid to be injected.

It would be desirable to have a portable hand piece for injecting a drug into the eye. Such a hand piece can include a limited reuse assembly attachable to and removable from a disposable tip segment. The disposable tip segment contains the drug, a needle for administering the drug, and a temperature control device, such as a heater, for altering the temperature of the drug. In order to protect medical personnel who are handling the two-part device, a protection or safety cap can be used to cover the needle end of the disposable tip segment. It would be desirable to have such a safety cap that is designed to be removable from the disposable tip segment to permit safe handling of the device.

### SUMMARY OF THE INVENTION

In one embodiment consistent with the principles of the present invention, the present invention is a keyed safety system for an injection device comprising a safety cap and a receptacle. The safety cap has a generally hollow body for receiving the needle end of an injection device. An orientation mechanism is disposed on the outer surface of the safety cap, a retention mechanism is disposed on the outer surface of the safety cap, and a shroud extends from the safety cap. The receptacle has a cavity for receiving the safety cap. A complimentary orientation mechanism is disposed on a surface of the cavity, and a complimentary retention mechanism is disposed on the surface of the cavity. The orientation mechanism on the safety cap is configured to engage the complimentary orientation mechanism on the cavity so that the safety cap fits into the cavity in a single orientation. The retention mechanism on the safety cap is configured to engage the complimentary retention mechanism on the cavity so that the safety cap is prevented from rotating in the cavity.

In another embodiment consistent with the principles of the present invention, the present invention is a keyed safety system for an injection device comprising a safety cap, a receptacle, and a disposable tip segment. The safety cap has a generally hollow body for receiving the needle end of the tip segment. A safety cap orientation mechanism is disposed on the outer surface of the safety cap, a safety cap retention mechanism is disposed on the outer surface of the safety cap, and a shroud extends from the safety cap. The receptacle has a cavity for receiving the safety cap. A complimentary orientation mechanism is disposed on a surface of the cavity, and a complimentary retention mechanism is disposed on the surface of the cavity. The disposable tip segment has a needle disposed on one end of the tip segment, a tip segment orientation mechanism disposed on an outer surface of the tip segment, and a tip segment retention mechanism disposed on a surface of the tip segment. The safety cap orientation mechanism is configured to engage the complimentary orientation mechanism on the cavity so that the safety cap fits into the cavity in a single orientation. The tip segment orientation mechanism is configured to engage the safety cap orientation mechanism so that the tip segment fits into the safety cap in a single orientation. The safety cap retention mechanism is configured to engage the complimentary retention mechanism on the cavity so that the safety cap is prevented from rotating in the cavity. The tip segment retention mechanism is configured to engage the safety cap retention mechanism so that the tip segment is prevented from rotating in the safety cap.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying figures, which are incorporated in and constitute a part of this specification, illustrate several embodiments of the invention and together with the description, serve to explain the principles of the invention.

FIG. 1 is a perspective view of a prior art syringe.

FIG. 2 is a view of an ophthalmic medical device including a disposable tip segment and a limited reuse assembly according to the principles of the present invention.

FIG. 3 is an embodiment of a limited reuse assembly according to the principles of the present invention.

FIG. 4 is a cross section view of a disposable tip segment and a limited reuse assembly according to the principles of the present invention.

FIG. 5 is a cross section view of a disposable tip segment according to the principles of the present invention.

FIG. 6 is a side view of a disposable tip segment and a safety cap according to the principles of the present invention.

FIG. 7 is a side view of a disposable tip segment and a safety cap according to the principles of the present invention.

FIG. 8 is a side view of a disposable tip segment and a safety cap according to the principles of the present invention.

FIG. 9 is a top view of a receptacle for the safety cap according to the principles of the present invention.

FIG. 10 is a side view of a receptacle for the safety cap according to the principles of the present invention.

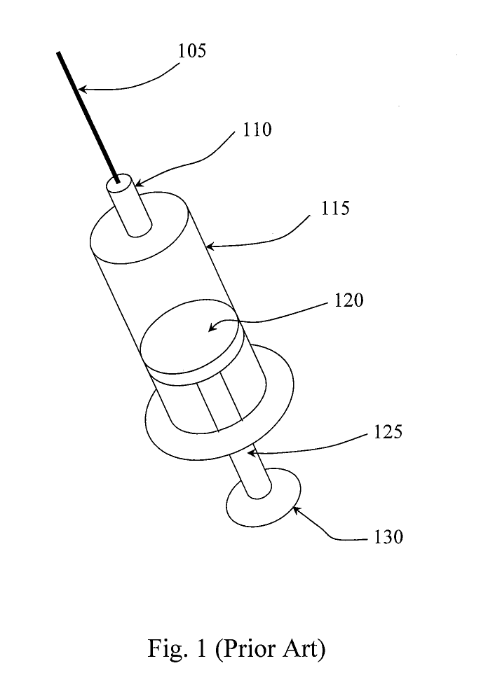

### DETAILED DESCRIPTION OF THE PREFERRED EMBODIMENTS

Reference is now made in detail to the exemplary embodiments of the invention, examples of which are illustrated in the accompanying figures. Wherever possible, the same reference numbers are used throughout the figures to refer to the same or like parts.

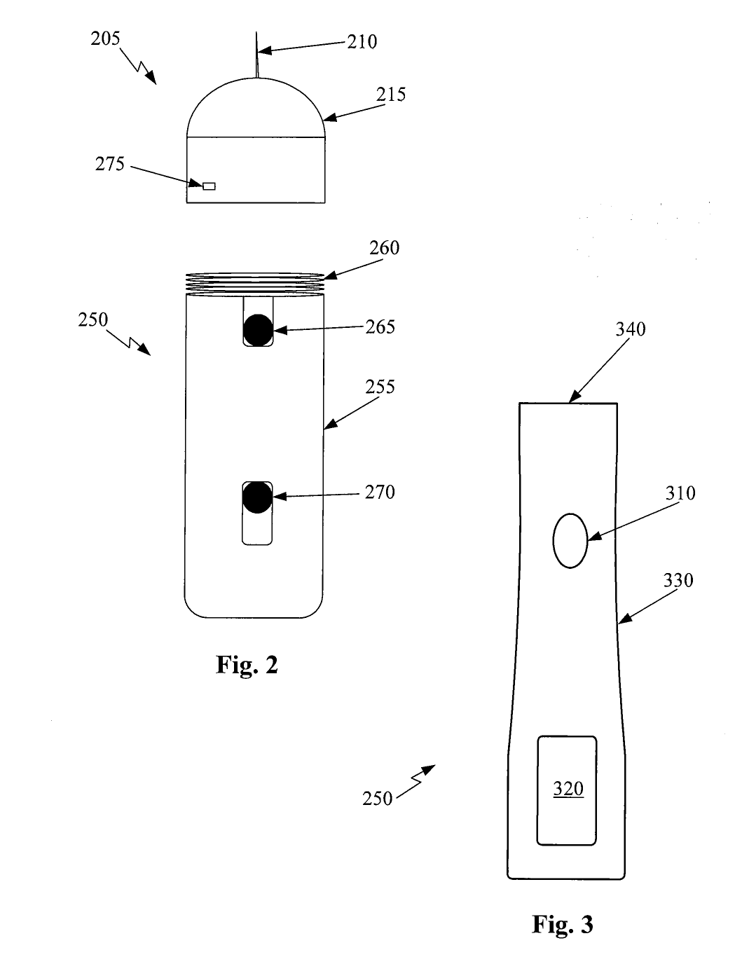

FIG. 2 depicts one view of an ophthalmic medical device including a disposable tip segment and a limited reuse assembly according to an embodiment of the present invention. In FIG. 2, the medical device includes a tip segment 205 and a limited reuse assembly 250. The tip segment 205 includes a needle 210, a housing 215, and an optional light 275. The limited reuse assembly 250 includes a housing 255, a switch 270, a lock mechanism 265, and a threaded portion 260.

Tip segment 205 is capable of being connected to and removed from limited reuse assembly 250. In this embodiment, tip segment 205 has a threaded portion on an interior surface of housing 215 that screws onto the threaded portion 260 of limited reuse assembly 250. In addition, lock mechanism 265 secures tip segment 215 to limited reuse assembly 250. Lock mechanism 265 may be in the form of a button, a sliding switch, or a cantilevered mechanism. Other mechanisms for connecting tip segment 205 to limited reuse assembly 250, such as those involving structural features that mate with each other, are commonly known in the art and are within the scope of the present invention.

Needle 210 is adapted to deliver a substance, such as a drug, into an eye. Needle 210 may be of any commonly known configuration. Preferably, needle 210 is designed such that its thermal characteristics are conducive to the particular drug delivery application. For example, when a heated drug is to be delivered, needle 210 may be relatively short (several millimeters) in length to facilitate proper delivery of the drug based on thermal characteristics.

Switch 270 is adapted to provide an input to the system. For example, switch 270 may be used to activate the system or to turn on a heater. Other switches, buttons, or user-directed control inputs are commonly known and may be employed with limited reuse assembly 250 and/or tip segment 205.

Optional light 275 is illuminated when tip segment 205 is ready to be used. Optional light 275 may protrude from housing 215, or it may be contained within housing 215, in which case, optional light 275 may be seen through a clear portion of housing 215. In other embodiments, optional light 275 may be replaced by an indicator, such as a liquid crystal display, segmented display, or other device that indicates a status or condition of disposable tip segment 205. For example, optional light 275 may also pulse on and off to indicate other states, such as, but not limited to a system error, fully charged battery, insufficiently charged battery or faulty connection between the tip segment 205 and limited use assembly 250. While shown on tip segment 205, optional light 275 or other indicator may be located on limited reuse assembly 250.

FIG. 3 is another embodiment of a limited reuse assembly according to the principles of the present invention. Limited reuse assembly 250 includes a button 310, a display 320, and a housing 330. Disposable tip segment 205 attaches to end 340 of limited reuse assembly 250. Button 310 is actuated to provide an input to the system. As with switch 270, button 310 may activate a heater or other temperature control device or initiate actuation of a plunger. Display 320 is a liquid crystal display, segmented display, or other device that indicates a status or condition of disposable tip segment 205 or limited reuse assembly 250.

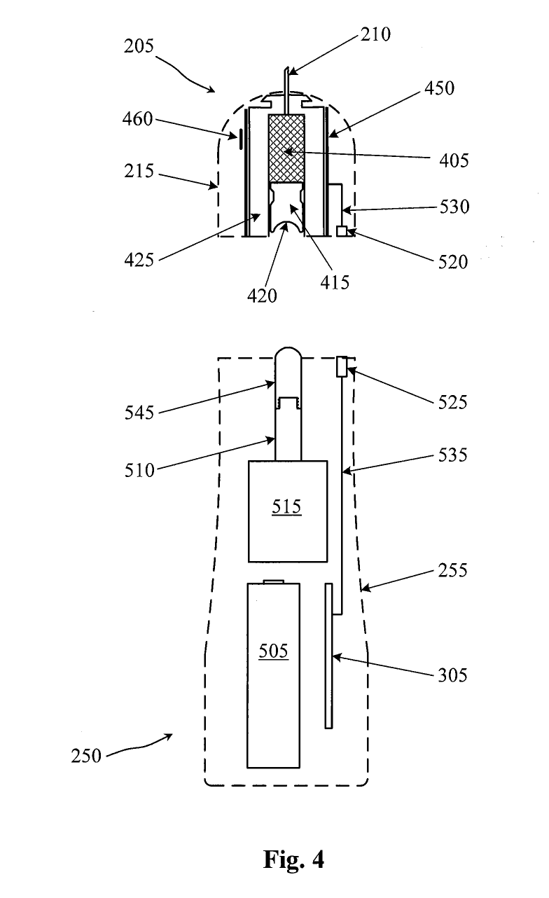

FIG. 4 is a cross section view of a disposable tip segment and a limited reuse assembly according to an embodiment of the present invention. FIG. 4 shows how tip segment 205 interfaces with limited reuse assembly 250. In the embodiment of FIG. 4, tip segment 205 includes plunger interface 420, plunger 415, dispensing chamber housing 425, tip segment housing 215, temperature control device 450, thermal sensor 460, needle 210, dispensing chamber 405, interface 530, and tip interface connector 520. Limited reuse assembly 250 includes mechanical linkage 545, actuator shaft 510, actuator 515, power source 505, controller 305, limited reuse assembly housing 255, interface 535, and limited reuse assembly interface connector 525.

In tip segment 205, plunger interface 420 is located on one end of plunger 415. The other end of plunger 415 forms one end of dispensing chamber 405. Plunger 415 is adapted to slide within dispensing chamber 405. An outer surface of plunger 415 is fluidly sealed to the inner surface of dispensing chamber housing 425. Dispensing chamber housing 425 surrounds the dispensing chamber 405. Typically, dispensing chamber housing 425 has a cylindrical shape. As such, dispensing chamber 405 also has a cylindrical shape.

Needle 210 is fluidly coupled to dispensing chamber 405. In such a case, a substance contained in dispensing chamber 405 can pass through needle 210 and into an eye. Temperature control device 450 at least partially surrounds dispensing chamber housing 425. In this case, temperature control device 450 is adapted to heat and/or cool dispensing chamber housing 425 and any substance contained in dispensing chamber 405. Interface 530 connects temperature control device 450 and thermal sensor 460 with tip interface connector 520.

The components of tip segment 205, including dispensing chamber housing 425, temperature control device 450, and plunger 415 are at least partially enclosed by tip segment housing 215. In one embodiment consistent with the principles of the present invention, plunger 415 is sealed to the interior surface of dispensing chamber housing 425. This seal prevents contamination of any substance contained in dispensing chamber 405. For medical purposes, such a seal is desirable. This seal can be located at any point on plunger 415 or dispensing chamber housing 425.

In limited reuse assembly 250, power source 505 provides power to actuator 515. An interface (not shown) between power source 505 and actuator 515 serves as a conduit for providing power to actuator 515. Actuator 515 is connected to actuator shaft 510. When actuator 515 is a stepper motor, actuator shaft 510 is integral with actuator 515. Mechanical linkage interface 545 is connected to actuator shaft 510. In this configuration, as actuator 515 moves actuator shaft 510 upward toward needle 210 mechanical linkage interface 545 also moves upward toward needle 210.

Controller 305 is connected via interface 535 to limited reuse assembly interface connecter 525. Limited reuse assembly interface connecter 525 is located on a top surface of limited reuse assembly housing 255 adjacent to mechanical linkage interface 545. In this manner, both limited reuse assembly interface connector 525 and mechanical linkage interface 545 are adapted to be connected with tip interface connector 520 and plunger interface 420 respectively.

Controller 305 and actuator 515 are connected by an interface (not shown). This interface (not shown) allows controller 305 to control the operation of actuator 515. In addition, an interface (not shown) between power source 505 and controller 305 allows controller 305 to control operation of power source 505. In such a case, controller 305 may control the charging and the discharging of power source 505 when power source 505 is a rechargeable battery.

Controller 305 is typically an integrated circuit with power, input, and output pins capable of performing logic functions. In various embodiments, controller 305 is a targeted device controller. In such a case, controller 305 performs specific control functions targeted to a specific device or component, such as a temperature control device or a power supply. For example, a temperature control device controller has the basic functionality to control a temperature control device. In other embodiments, controller 305 is a microprocessor. In such a case, controller 305 is programmable so that it can function to control more than one component of the device. In other cases, controller 305 is not a programmable microprocessor, but instead is a special purpose controller configured to control different components that perform different functions. While depicted as one component, controller 305 may be made of many different components or integrated circuits.

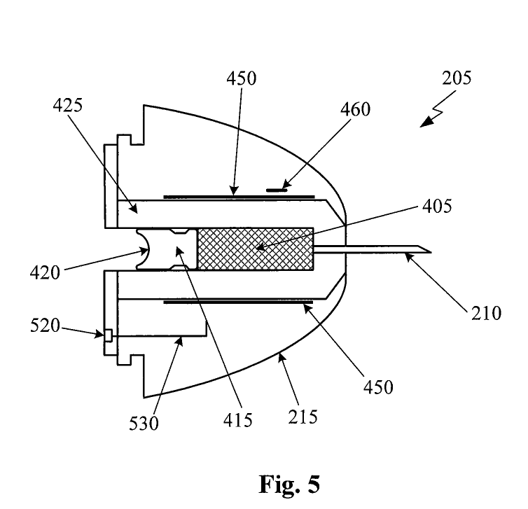

Tip segment 205 is adapted to mate with or attach to limited reuse assembly 250 as previously described. In the embodiment of FIG. 5, plunger interface 420 located on a bottom surface of plunger 415 is adapted to mate with mechanical linkage interface 545 located near a top surface of limited reuse assembly housing 255. In addition, tip interface connector 520 is adapted to connect with limited reuse assembly interface connector 525. When tip segment 205 is connected to limited reuse assembly 250 in this manner, actuator 515 and actuator shaft 510 are adapted to drive plunger 415 upward toward needle 210. In addition, an interface is formed between controller 305 and temperature control device 450. A signal can pass from controller 305 to temperature control device 450 through interface 535, limited reuse assembly interface connector 525, tip interface connector 520, and interface 530.

In operation, when tip segment 205 is connected to limited reuse assembly 250, controller 305 controls the operation of actuator 515. Actuator 515 is actuated and actuator shaft 510 is moved upward toward needle 210. In turn, mechanical linkage interface 545, which is mated with plunger interface 420, moves plunger 415 upward toward needle 210. A substance located in dispensing chamber 405 is then expelled through needle 210.

In addition, controller 305 controls the operation of temperature control device 450. Temperature control device 450 is adapted to heat and/or cool dispensing chamber housing 425. Since dispensing chamber housing 425 is at least partially thermally conductive, heating or cooling dispensing chamber housing 425 heats or cools a substance located in dispensing chamber 405. Temperature information can be transferred from thermal sensor 460 to controller 305 via any of a number of different interface configurations. This temperature information can be used to control the operation of temperature control device 450. When temperature control device 450 is a heater, controller 305 controls the amount of current that is sent to temperature control device 450. The more current sent to temperature control device 450, the hotter it gets. In such a manner, controller 305 can use a feed back loop utilizing information from thermal sensor 460 to control the operation of temperature control device 450. Any suitable type of control algorithm, such as a proportional integral derivative (PID) algorithm, can be used to control the operation of temperature control device 450.

FIG. 5 is a cross section view of a disposable tip segment for an ophthalmic medical device according to an embodiment of the present invention. In FIG. 5, disposable tip segment 205 includes housing 215, needle 210, plunger 415, plunger interface 420, dispensing chamber 405, dispensing chamber housing 425, temperature control device 450, thermal sensor 460, interface 530, and tip interface connector 520. Disposable tip segment 205 operates as a disposable injection device.

In the embodiment of FIG. 5, plunger 415 is located in dispensing chamber housing 425. Dispensing chamber 405 is enclosed by dispensing chamber housing 425 and plunger 415. Plunger 415 forms a fluid seal with the interior surface of dispensing chamber housing 425. Needle 210 is fluidly coupled to dispensing chamber 405. In this manner, a substance located in dispensing chamber 405 can be contacted by plunger 415 and pushed out of needle 210. Temperature control device 450 is located adjacent to dispensing chamber housing 425 and at least partially surrounds dispensing chamber 405. Housing 215 forms an outer skin on disposable tip segment 205.

In various embodiments of the present invention, temperature control device 450 is a heating and/or a cooling device. Temperature control device 450 is in thermal contact with dispensing chamber housing 425. As such, temperature control device 450 is capable of changing the temperature of the substance in dispensing chamber 405. Interface 530 and tip interface connector 520 couple temperature control device 450 to a limited reuse assembly. In such a case, temperature control device 450 can be powered and controlled by the limited reuse assembly. In one embodiment of the present invention, temperature control device 450 receives voltage via interface 530 from a limited reuse assembly. Providing a positive voltage across the temperature control device 450 causes it to produce heat. Providing a negative voltage across the temperature control device 450 causes it to cool.

A substance to be delivered into an eye, typically a drug, is located in dispensing chamber 405. In this manner, the substance is contacted by the inner surface of dispensing chamber housing 425 and one face of plunger 415. Typically, dispensing chamber 405 is cylindrical in shape. Temperature control device 450 is in thermal contact with dispensing chamber housing 425. In this manner, temperature control device 450 is adapted to control the temperature of the contents of dispensing chamber 425. Thermal sensor 460 provides temperature information to assist in controlling the operation of temperature control device 450.

In one embodiment of the present invention, the substance located in dispensing chamber 405 is a drug that is preloaded into the dispensing chamber. In such a case, disposable tip segment 205 is appropriate as a single use consumable product. Such a disposable product can be assembled at a factory with a dosage of a drug installed.

When a drug is preloaded into dispensing chamber 405, a set quantity of the drug can be preloaded. For example, 100 microliters of a drug can be loaded into dispensing chamber 405, and any quantity up to 100 microliters can be dispensed. In such a case, the plunger 415 can be moved a precise distance to deliver a precise dosage of drug from the dispensing chamber 405, through the needle 210, and into an eye. This provides for flexibility of dosing and for ease of assembly.

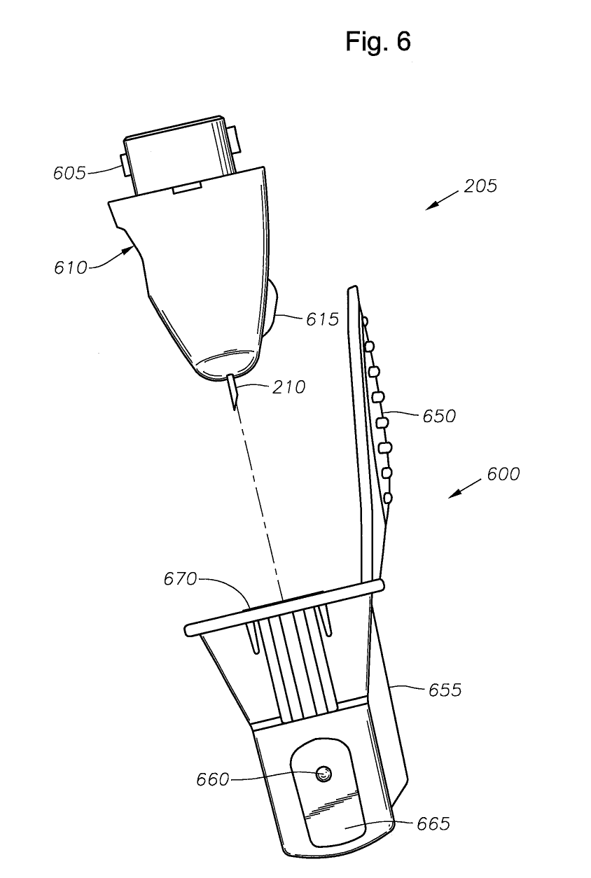

FIG. 6 is a side view of a disposable tip segment and a safety cap according to the principles of the present invention. Disposable tip segment 205 includes locking tab 605, surface 610, rib 615, and needle 210. Safety cap 600 includes activation button shroud 650, rib 655, retention dimple 660, flat face 665, and locking mechanism 670. Disposable tip segment 205 is configured to fit into safety cap 600 in the orientation shown in FIG. 6.

In tip segment 205, locking tab 605 (and a second locking tab shown opposite locking tab 605) is configured to mate with slots in a limited reuse assembly 250. Preferably, tip segment 205 is configured to be inserted into a limited reuse assembly 205 and rotated (preferably about ⅛ turn) so that tip segment 205 is securely attached to limited reuse assembly 250. In this manner, locking tab 605 is inserted into a slot in limited reuse assembly 250 that extends circumferentially at least a portion of the way around an interior surface of limited reuse assembly 250. As such, tip segment 205 is rotated and locked onto limited reuse assembly 250.

Surface 610 is shaped such that it interfaces with a similarly shaped surface (not shown) on the interior of safety cap 600. In one embodiment of the present invention, surface 610 is a flat surface that rests against a similarly flat surface, orientation indent 850 on the interior of safety cap 600. In this manner, tip segment 205 is keyed to fit into safety cap 600 in one orientation.

Similarly, rib 615 on tip segment 205 may be adapted to fit on the underside of rib 655 on safety cap 600. In this manner, at least a part of the interior of rib 655 is hollow so that it receives rib 615. As such, tip segment 205 is keyed to fit into safety cap 600 in one orientation. While both a rib 615 and a surface 610 are depicted, either one may be present without the other one being present. In other embodiments of the present invention, various shapes or surfaces on tip segment 205 are configured to fit with complimentary shapes or surfaces in safety cap 600.

Activation button protection shroud 650 prevents an activation button (like button 310) from being depressed while safety cap 600 is on tip segment 205. In this manner, an injection cannot be initiated until the safety cap is removed.

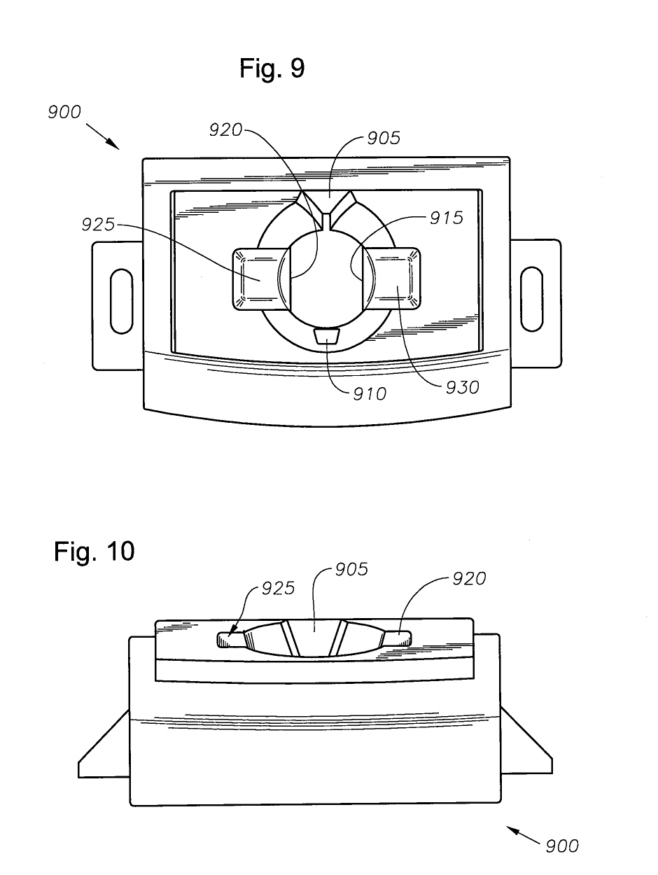

Rib 655 is designed to fit into recess 905 of receptacle 900 shown in FIG. 9. In this manner, safety cap 600 fits into receptacle 900 only in one orientation. As such, safety cap 600 is keyed to receptacle 900. A retention dimple 660 is provided so that safety cap 600 snaps into receptacle 900. Retention dimple 660 is designed to provide a retaining force on safety cap 600 that tends to hold safety cap 600 in receptacle 900.

Surface 665 & 780 are configured to rest against a complimentary surface 915 & flat surface 920 in receptacle 900. While in FIGS. 6-9, these surfaces are depicted as being flat, they may be of any shape. Surfaces 665 & 780 and 915 & 920 provide a retention force that holds safety cap 600 (and tip segment 205) in place while a limited reuse assembly 250 is rotated with respect to tip segment 205. In this manner, a limited reuse assembly can be rotated on or off a tip segment 205 (with a safety cap 600) located in receptacle 900. This configuration allows for a tip segment 205 to be removed from a limited reuse assembly 250 by means of a simple rotating movement. The used tip segment 205 can then be removed from the receptacle 900, for example, by lifting up on the activation button shroud 650. In this manner, tip segment 205 (with safety cap 600 in it) can be discarded without needle 210 being exposed.

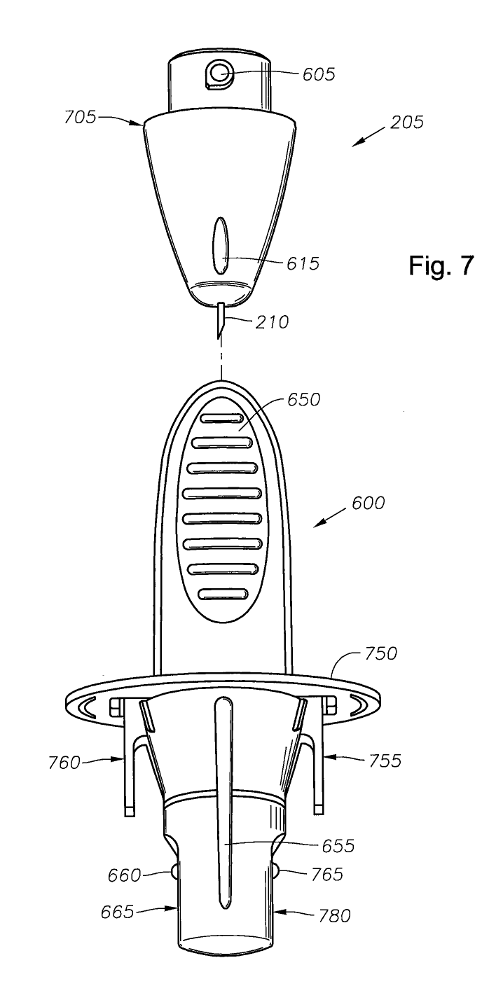

FIG. 7 is a side view of a disposable tip segment and a safety cap according to the principles of the present invention. Disposable tip segment 205 includes locking tab 605, rib 615, groove 705, and needle 210. Safety cap 600 includes activation button shroud 650, rib 655, retention dimples 660 and 765, shroud 750, and release tabs 755 and 760. Disposable tip segment 205 is configured to fit into safety cap 600 in the orientation shown in FIG. 7.

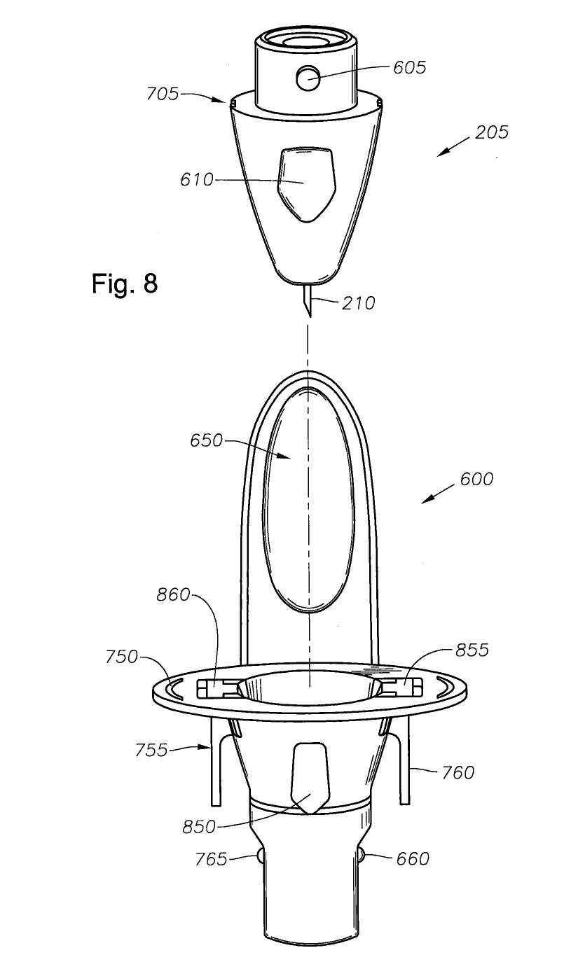

In tip segment 205, groove 705 is configured to be engaged by grippers 855 and 860 (of FIG. 8). Groove 705 may extend circumferentially around tip segment 205 or groove 705 may be segmented so that a portion of groove 705 exists on both sides of disposable tip segment 205 as shown in FIG. 7. Groove 705 provides a mechanism for securing safety cap 600 (through grippers 855 and 860) to tip segment 205.

Safety cap 600 has a shroud 750 that extends around a periphery of the hollow cavity that receives tip segment 205. This shroud 750 provides additional protection from an unintended needle stick. A medical professional who holds safety cap 600 (by the end on which rib 655 is present) is further protected by shroud 750 from a needle stick.

Release tabs 755 and 760 can be pressed inward toward the body of safety cap 600 to release grippers 855 and 860. In this manner, release tabs 755 and 760 act like levers. Release tabs 755 and 760 are biased such that safety cap 600 is secured to tip segment 205 when safety cap 600 is on tip segment 205.

FIG. 8 is a side view of a disposable tip segment and a safety cap according to the principles of the present invention. Disposable tip segment 205 includes locking tab 605, surface 610, groove 705, and needle 210. Safety cap 600 includes activation button shroud 650, tab 850, retention dimples 660 and 765, shroud 750, release tabs 755 and 760, and grippers 855 and 860. Disposable tip segment 205 is configured to fit into safety cap 600 in the orientation shown in FIG. 8.

Tab 850 in safety cap 600 is designed to fit with surface 610 of tip segment 205. Tab 850 extends into the interior cavity of safety cap 600. In this manner, a protrusion with a surface on the interior of safety cap 600 (formed by tab 850) provides one way of keying tip segment 205 to safety cap 600 (so that tip segment 205 fits into safety cap 600 in one orientation).

Grippers 855 and 860, in conjunction with release tabs 755 and 760, act to secure safety cap 600 to tip segment 205. Teeth on the end of grippers 855 and 860 are designed to fit into groove 705 and to hold safety cap 600 securely on tip segment 205. Depressing release tabs 755 and 760 inward towards the body of safety cap 600 (lever action) releases grippers 855 and 860 from groove 705 so that safety cap 600 can be removed from tip segment 205.

FIG. 9 is a top view of a receptacle for the safety cap according to the principles of the present invention. FIG. 10 is a side view of a receptacle for the safety cap according to the principles of the present invention. Safety cap 600 is designed to be received in receptacle 900. Receptacle 900 has rib recess 905, protrusion 910, surfaces 915 and 920, and release tab nests 925 and 930.

Rib recess 905 is configured to receive rib 655 of safety cap 600. Rib recess 905 (when engaged with rib 655) acts as a key so that safety cap 600 can be inserted into receptacle 900 in one orientation. In addition, rib recess 905 (when engaged with rib 655) provides a retention force when a limited reuse assembly is rotated to engage a tip segment 205 that rests in a safety cap 600 in receptacle 900. Likewise, protrusion 910 is configured to fit with tab 850. As such, protrusion 910 (when engaged with tab 850) provides a keying function and a retention function. Protrusion 910 (when engaged with tab 850) acts as a key so that safety cap 600 can be inserted into receptacle 900 in one orientation. In addition, protrusion 910 (when engaged with tab 850) provides a retention force when a limited reuse assembly is rotated to engage a tip segment 205 that rests in a safety cap 600 in receptacle 900.

Surfaces 915 and 920 provide a similar retention function. Surfaces 915 and 920 are configured to rest against surfaces 665 and 780. Surface 665 is configured to rest against a complimentary surface 915 in receptacle 900. While in FIGS. 6-9, these surfaces are depicted as being flat, they may be of any shape. Surfaces 665 and 780 on safety cap 600 and surfaces 915 and 920 on receptacle 900 provide a retention force that holds safety cap 600 (and tip segment 205) in place while a limited reuse assembly 250 is rotated with respect to tip segment 205. In this manner, a limited reuse assembly can be rotated on or off a tip segment 205 (with a safety cap 600) located in receptacle 900. This configuration allows for a tip segment 205 to be removed from a limited reuse assembly 250 by means of a simple rotating movement. The used tip segment 205 can then be removed from the receptacle 900, for example, by lifting up on the activation button shroud 650. In this manner, tip segment 205 (with safety cap 600 on it) can be discarded without needle 210 being exposed.

In addition, surfaces 915 and 920 may also include detents to engage retention dimples 660 and 765. In this manner, when safety cap 600 is placed in receptacle 900, the detents are engaged with retention dimples 660 and 765 to provide a retention force that holds safety cap 600 in receptacle 900.

Release tab nests 930 and 925 may engage release tabs 755 and 760 so that safety cap 600 can be removed from tip segment 205. In another configuration according to the principles of the present invention, a medical professional can remove safety cap 600 by depressing release tabs 755 and 760.

The safety cap system described can be operated in numerous different ways to provide protection against needle stick. In one way, a tip segment 205 comes packaged with a safety cap 600 covering tip segment 205. A medical professional can attach tip segment 205 (with safety cap 600 on it) to a limited reuse assembly by hand. For example, placing tip segment 205 (with safety cap 600 in place) on a limited reuse assembly and turning tip segment 205 and safety cap 600 secures tip segment 205 to a limited reuse assembly. The professional can then remove the safety cap 600 by placing it into receptacle 900. Alternatively, the professional can depress release tabs 755 and 760 to remove safety cap 600 (which can then be placed in receptacle 900). After an injection is completed, tip segment 205 (with the limited reuse assembly still attached) can be placed in safety cap 600 (which rests in receptacle 900. The limited reuse assembly can be turned to disengage tip segment 205 from it. After the limited reuse assembly is removed from tip segment 205, both tip segment 205 and safety cap 600 can be discarded.

While the present invention is described in the context of a single-use ophthalmic drug delivery device, the present invention encompasses any medical device or injection device. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A keyed safety system for an injection device comprising:
a safety cap and a receptacle;
the safety cap comprising a generally hollow body for receiving the needle end of an injection device;
an orientation mechanism disposed on the outer surface of the safety cap;
a retention mechanism disposed on the outer surface of the safety cap; and
a shroud extending from the safety cap;
the receptacle comprising a cavity for receiving the safety cap;
a complimentary orientation mechanism disposed on a surface of the cavity; and
a complimentary retention mechanism disposed on the surface of the cavity;
wherein the orientation mechanism on the safety cap is configured to engage the complimentary orientation mechanism on the cavity so that the safety cap fits into the cavity in a single orientation;
and further wherein the retention mechanism on the safety cap is configured to engage the complimentary retention mechanism on the cavity so that the safety cap is prevented from rotating in the cavity.

2. The system of claim 1 wherein the orientation mechanism is a rib and the complimentary orientation mechanism is a recess for holding the rib.

3. The system of claim 1 wherein the orientation mechanism is a first surface and the complimentary orientation mechanism is a second surface configured to fit with the first surface.

4. The system of claim 1 wherein the retention mechanism is a first surface and the complimentary retention mechanism is a second surface configured to fit with the first surface so as not to allow rotation of the safety cap in the receptacle.

5. The system of claim 1 wherein the retention mechanism is a retention dimple and the complimentary retention mechanism is a recessed surface.

6. The system of claim 1 wherein the shroud is configured to shield an activation button to prevent unwanted activation of the injection device.

7. The system of claim 1 wherein the shroud is configured to further prevent unwanted needle stick when the safety cap is placed on an injection device.

8. The system of claim 1 further comprising a pair of release tabs for securing the safety cap to a tip segment.

9. A keyed safety system for an injection device comprising:
a safety cap, a receptacle, and a disposable tip segment;
the safety cap comprising a generally hollow body for receiving the needle end of the tip segment; a safety cap orientation mechanism disposed on the outer surface of the safety cap; a safety cap retention mechanism disposed on the outer surface of the safety cap; and a shroud extending from the safety cap;
the receptacle comprising a cavity for receiving the safety cap; a complimentary orientation mechanism disposed on a surface of the cavity; and a complimentary retention mechanism disposed on the surface of the cavity;
the disposable tip segment comprising a needle disposed on one end of the tip segment, a tip segment orientation mechanism disposed on an outer surface of the tip segment, and a tip segment retention mechanism disposed on a surface of the tip segment;
wherein the safety cap orientation mechanism is configured to engage the complimentary orientation mechanism on the cavity so that the safety cap fits into the cavity in a single orientation;
further wherein the tip segment orientation mechanism is configured to engage the safety cap orientation mechanism so that the tip segment fits into the safety cap in a single orientation;
further wherein the safety cap retention mechanism is configured to engage the complimentary retention mechanism on the cavity so that the safety cap is prevented from rotating in the cavity;
and further wherein the tip segment retention mechanism is configured to engage the safety cap retention mechanism so that the tip segment is prevented from rotating in the safety cap.

10. The system of claim 9 wherein the safety cap orientation mechanism is a rib and the complimentary orientation mechanism is a recess for holding the rib.

11. The system of claim 9 wherein the safety cap orientation mechanism is a first surface and the complimentary orientation mechanism is a second surface configured to fit with the first surface.

12. The system of claim 9 wherein the safety cap retention mechanism is a first surface and the complimentary retention mechanism is a second surface configured to fit with the first surface so as not to allow rotation of the safety cap in the receptacle.

13. The system of claim 9 wherein the safety cap retention mechanism is a retention dimple and the complimentary retention mechanism is a recessed surface.

14. The system of claim 9 wherein the shroud is configured to shield an activation button to prevent unwanted activation of the injection device.

15. The system of claim 9 wherein the shroud is configured to further prevent unwanted needle stick when the safety cap is placed on an injection device.

16. The system of claim 9 wherein the safety cap orientation mechanism is a hollow rib and the tip segment orientation mechanism is a rib configured to fit in the hollow rib.

17. The system of claim 9 wherein the safety cap orientation mechanism is a first surface and the tip segment orientation mechanism is a second surface configured to fit with the first surface.

18. The system of claim 9 wherein the safety cap retention mechanism is a first surface and the complimentary retention mechanism is a second surface configured to fit with the first surface so as not to allow rotation of the tip segment in the safety cap when a toque is applied to twist the tip segment off while engaged within the safety cap that is nested in the receptacle.

19. The system of claim 9 further comprising a pair of release tabs for securing the safety cap to the tip segment.
